(19) United States
(12) Patent Application Publication
(10) Pub. No.: US 2011/0112184 A1
(43) Pub. Date: May 12, 2011

(54) COMPOUNDS WITH GLYCIDIC STRUCTURE ACTIVE IN THE THERAPY OF SYSTEMIC AND LOCAL INFLAMMATION

(76) Inventors: Barbara La Perla, Milano (IT); Francesco Nicotra, Milano (IT); Andrea Balsari, Milano (IT); Marco Palazzo, Besozzo (IT); Cristiano Rumio, Milano (IT)

(21) Appl. No.: 12/991,455

(22) PCT Filed: May 7, 2009
(86) PCT No.: PCT/EP2009/003267

§ 371 Date: Jan. 17, 2011

(30) Foreign Application Priority Data
May 9, 2008 (IT) MI2008A000846

(51) Int. Cl. A61K 31/351; C07D 315/00; C07D 311/00; A61K 31/35; A61P 3/10

(52) U.S. Cl. 514/456; 549/417; 549/396; 514/460

(57) ABSTRACT

Compounds of the general formula (I) and (II) (including formulae (III) to (V)), wherein X=—CH₂—, —O—, —S— n=0-10 Y=—NH—, —NHSO₂—, —NHSO—, —NHCO—, —S—, —O—, —CH═CH—R₁-R₇, equal or different can be hydrogen, alkyl C₁-C₄, alkenyl C₂-C₄, cycloalkyl C₃-C₇, aryl or heteroaryl; may be substituted with one or more alkyl C₁-C₄, alkoxyl C₁-C₄, alkylthio C₁-C₄ or halogens; —NR₈R₉, where R₈ e R₉, equal or different, represent hydrogen, alkyl C₁-C₄, alkenyl C₂-C₄, cycloalkyl C₃-C₇, aryl or heteroaryl, may be substituted with one or more C₁-C₄, alkoxyl C₁-C₄, alkylthio C₁-C₄ or halogens; —(CH₂)ₙ, —COOR₁₀, with n′=0-4 and R₃=hydrogen or alkyl C₁-C₄, and their addition salts with organic and inorganic acids, or alkaline and alkaline earth metal or ammonium ions, with the exclusion of the compounds having the following formulae ((VI) to (IX)).

The present invention concerns novel compounds with glycidic structure for the therapy of inflammatory diseases, endotoxic shock, allergic inflammation, chronic inflammation of the gut, for the protection against mucositis induced by cytotoxic drugs or radiotherapy as well as for the therapy of type II diabetes.

### BACKGROUND OF THE INVENTION

### Inflammatory Pathologies and Current Therapies

Inflammation is a non-specific innate defense mechanism, that can be activated by a large number of dangerous events of various kinds, including chemical and biological agents. Elimination of the causes and consequences of these events, i.e. cell and tissue damages, is the goal of inflammation.

In the absence of inflammation, wounds and infections would never heal and progressive destruction of the tissue would compromise the survival of the organism. However, an inflammation that runs unchecked can also lead to a host of inflammatory diseases, such as hay fever, atherosclerosis, and rheumatoid arthritis. Finally, prolonged inflammation leads to a progressive shift in the type of cells which are present at the site of inflammation and is characterized by simultaneous destruction and healing of the tissue from the inflammatory process.

Some examples of inflammatory diseases that can be therapeutic targets for compounds with the general structure I or II, are reported below.

Luminal Bacterial Infections and Diarrhea

The presence of pathogenic agents in the intestinal lumen can induce an inflammatory state with consequent onset of intestinal inflammatory diseases. This intestinal pathology may have two different etiologies. Some pathogenic agents possess virulence factors that allow them to cross the epithelial barrier of enterocytes, thereby compromising its protective role in innate immunity. On the other hand, other pathogens can induce an inflammatory state without crossing the epithelial barrier. Increased production of cytokines from epithelial cells, weight loss, activation of transcription factors like NF-kB, and signs of suffering of epithelial cells and, eventually, apoptosis, are markers of luminal bacterial infection.

Currently, one of the most important therapeutic goals in these settings is to reduce as vigorously as possible dehydration; it is necessary to reintegrate liquids, mineral salts and sugars with food and electrolytic solutions by intravenous administration. Most importantly, however, is the use of antibiotic therapy.

Sepsis

Sepsis is the most frequent cause of death in intensive care units: antibiotic therapy is of little use in this pathology, and may even be of harm by inducing bacterial disintegration, and consequent release of endotoxins, that may exacerbate shock. The management of severe sepsis rests on four key interventions: control of infection, hemodynamic support, immunomodulatory approaches, and metabolic/endocrine support.

Asthma

Asthma is an inflammatory disease involving the respiratory system, often in response to one or more allergenic triggers that recruit various types of cells like mastocytes, eosinophils and T-lymphocytes, which play important roles in the pathogenesis. Asthma leads to a variable and partially reversible obstruction to air flow, and is accompanied by overdeveloped mucus glands, airway thickening due to scarring and inflammation. Bronchoconstriction, is due to narrowing of the airways in the lungs as a consequence of tightening of the surrounding smooth muscle. It is also due to oedema and swelling caused by an immune response to allergens.

Key drugs for anti-inflammatory therapy of asthma are aerosolized corticosteroids. At therapeutic dosages, side effects of these drugs are dysphonia and oropharyngeal candidiasis caused by in situ drug deposition. Another important drug category for the treatment of asthma is represented by long- or short-lasting bronchodilators.

Crohn's Disease

Crohn's disease is an autoimmune-like disorder characterized by chronic, idiopathic inflammation of the intestinal mucosal tissue, which causes a range of symptoms including abdominal pain, severe diarrhoea, rectal bleeding and wasting. This pathology is associated with overproduction of proinflammatory cytokines like IL-12, IL-17 and IFN-γ, caused by an hyperresponsiveness of the immune system at the level of the intestinal mucosa. Crohn's disease induces abnormal functioning of intercellular junctions and initiates a mucosal inflammatory disorder that culminates with increased paracellular permeability with consequent loss of the epithelial barrier function. This event leads to diffusion of the luminal content in the interstitial spaces with an increased inflammatory response.

Corticosteroids are key drugs also for the therapy of Crohn's disease. Metronidazole, which acts against some types of bacteria and parasites, is effective against anal and perineal lesions. The mechanism of action of metronidazole in this setting is unknown. One of the most important side effect of metronidazole, if used over a long time, is carcinogenicity. (Rossini A, Rumio C, Sfondrini L, Tagliabue E, Miceli R, Mariani L. Palazzo M, Ménard S, Balsari A. Influence of antibiotic treatment on breast carcinoma development in proto-neu transgenic mice. Cancer Res. 2006 Jun. 15; 66(12):6219-24).

Mucositis Caused by Drugs Therapies and Radiotherapy

Mucositis consists of tumefaction, irritation and ulceration of epithelial cells of the gastrointestinal tract. Any part of this tract can be affected. Mucositis is generally the consequence of chemotherapeutic and/or radiotherapeutic treatments that preferentially act against rapidly proliferating cells, like the epithelial cells of the gastrointestinal tract. This can cause irritation, inflammation and loss of the barrier effect of the gastrointestinal mucosa and in the worst case it may lead to loss of the integrity of the epithelial barrier.

Current therapies for mucositis of limited use, and they impact on the etiology of the disease; in most cases, only generic anti-inflammatory drugs are used. Oral and gastrointestinal mucositis are treated with rehydratation, washes with solutions containing soda and clorexin. Local (lidocain 2%, for example) and systemic anaesthetic drugs are also used. Another approach takes advantages of products like poly-vinyl pyrrolidone) and hyaluronic acid, which provide mechanical protection against the lesions.

Diabetes Mellitus

Type 2 diabetes, or non-insulin-dependent diabetes mellitus, is the most frequent form of diabetes. In its initial phases, insulin production is normal or increased. Subsequently, it production progressively declines, because of exhaustion of the insulin-producing β-cells of the Langerhans islets of the pancreas. One of the mainstays of the therapy of type 2 diabetes are drugs having hypoglicemic effects, which are employed once dietary intervention and increased physical activity have failed to achieve satisfactory glycemic control.

In the Italian patent application MI2006A002165, filed on 13 Nov. 2006, the use of glucose as an anti-inflammatory molecule, when used orally at high doses (10 g/Kg) has been proposed. This inhibition has been found to be mediated by the sodium-dependent glucose transporter-1 (SGLT-1). The main drawbacks of this treatment are the high doses of glucose that must be administered to achieve protection, and the hyperglycemic consequences of this approach. Therefore, the identification of glucomimetics able to induce anti-inflammatory effects upon binding to SGLT-1 at pharmacological concentrations would be an important target.

To our knowledge, no examples of molecules inducing anti-inflammatory effects through binding to SGLT-1 are reported in the literature, whereas some aryl β-glucosides have been patented as SGLT-1 inhibitors [Yonekubo (2006), Eckhardt (2006), Nomura (2005), Fujikura (2005), Fushimi-Teranishi (2005), Fushimi-Fujikura (2005), Nomura-Kawanishi (2005)]. Moreover, very little is known on the structure of this transporter and its mechanism [Silverman (1998), Wright (2001), Raja (2003, 2004), Xia (2004).

### DESCRIPTION OF THE INVENTION

The present invention provides compounds of general formulae I and II

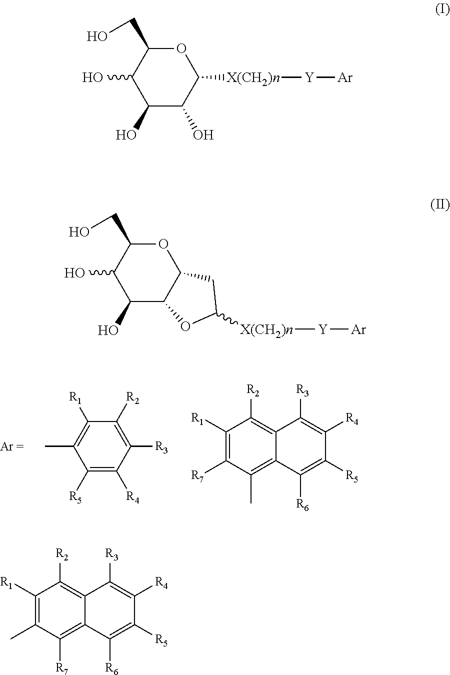

wherein

X=—CH₂—, —O—, —S—

n=0-10

Y=—NH—, —NHSO₂—, —NHSO—, —NHCO—, —S—, —O—, —CH═CH—

R₁-R₇, which can be the same or different, represent

hydrogen, alkyl C₁-C₄, alkenyl C₂-C₄, cycloalkyl C₃-C₇, aryl or heteroaryl,

which may be substituted with one or more alkyl C₁-C₄, alkoxyl C₁-C₄, alkylthio C₁-C₄ or halogens;

—NR₈R₉, where R₈ and R₉, equal or different, represent hydrogen, alkyl C₁-C₄, alkenyl C₂-C₄, cycloalkyl C₃-C₇, aryl or heteroaryl,

may be substituted with one or more alkyl C₁-C₄, alkoxyl C₁-C₄, alkylthio C₁-C₄ or halogens;

—(CH₂)ₙ—COOR₃, with n′=0-4 and R₃=hydrogen or alkyl C₁-C₄,

and their enantiomers, their diastereoisomers, their addition salts with organic and inorganic acids, or their alkaline and alkaline earth metal or their ammonium ions,

with the exclusion of the compounds having the following formulae:

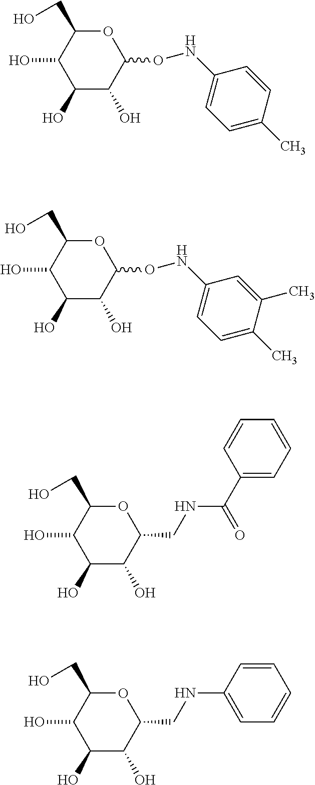

The disclaimed compounds are known from:

1) Hakamata, J. Appl. Glycoscience, 2006, 53, 149-54; Id. Bioorg Med Chem. Lertt. 2005, 23, 27-39; Id. J. Carbohydr. Chem. 2004, 23, 27-39

2) Dietrich, Carb. Res. 1993, 250, 161-176

3) Inoue, Nippon Nogel Kagaku Kaishi, 1948, 22, 70-1

4) Japanese patent N. 172795.

The invention also concerns the use of compounds of general formula (I) and (II) for the therapy of inflammatory diseases, such as bacterial inflammation at the level of intestinal lumen, endotoxic shock, allergic inflammation, chronic inflammation of the gut, for the protection against mucositis induced by therapy with cytotoxic drugs or radiotherapy. Another use of the mentioned compounds, according to the invention, is to limit absorption of glucose at the intestinal level. This effect is of particular interest for the therapy of type II diabetes.

We have unexpectedly found that molecules containing a glycosidic portion, of formula (I) and (II), are able to exert the same functions of glucose but at much lower concentrations, namely 100.000/1.000.000 lower, than glucose.

A further embodiment of the invention is therefore represented by pharmaceutical compositions for use in the treatment of inflammatory diseases and diabetes, containing one or more compounds of formula (I) or (II), in combination with one or more inert, non-toxic pharmaceutically acceptable carriers, optionally associated with others compatible drugs, for example glutamine.

The preferred compound of the invention are those wherein X represents a CH₂. Advantageously the invention relates to compounds of formula (I) and (II) wherein n is equal to 0-5. Preferably, the invention relates to compounds of formula (I) and (II) wherein Y is —NHSO₂— and NHCO—. The preferred compounds of the invention are those wherein R₁-R₇, which can be the same or different, represent a hydrogen atom or —NR₈R₉ where R₈ and R₉ equal of different, represent preferably alkyl C₁-C₄.

In particular, α-C-glucopyranosides in which a naphthyl substituent is linked to a short anomeric moiety, like 5-(dimethylamino)-N-[2′-(α-D-glucopyranosyl)ethyl]-1-naphthalene sulfonamide (4), N-[2′-(α-D-glucopyranosyl)ethyl]-1-naphthalene sulfonamide (5), N-[2′-(-(α-D-glucopyranosyl)ethyl]-1-naphthalenecarboxyamide (6), 5-(dibutylamino)-N-[2∝-(α-D-glucopyranosyl)ethyl]-1-naphthalene sulfonamide (7), 5-(dimethylamino)-N-[4′-(α-D-glucopyranosyl)buthyl]-1-naphthalenesulfonamide (14) and 5-(dimethylamino)-N-[2′-(α-D-galactopyranosyl)ethyl]-1-naphthalenesulfonamide (4′) are described in the present invention.

Compounds 4, 5, 6, 7 and 4′ have been synthesized as illustrated in Scheme 1.

A solution of allyl C-glycosides 1/1′, obtained through a stereoselective allylation according to a procedure described by Gray (Bennek, 1987), in MeOH/H₂O is cooled to −78° C. and saturated with ozone. After 75 min the pale blue solution is saturated with oxygen then with nitrogen till colorless. Then dimethylsulfide (Me₂S) is added and the solution is left warming at room temperature under stirring for 12 h. After removal of the solvent under reduced pressure, compounds 2/2′ are obtained as diastereoomeric mixtures of bicyclic semiacetals. The crudes 2/2′ are then subjected to reductive amination with anhydrous ammonium acetate (5 equivalents) and NaCNBH₃, thus yielding amines 3/3′. Compounds 4, 5, 6, 7 and 4′ are then obtained using the following procedure: amine 3/3′ is dissolved in dry MeOH (0.2 mmol/ml) and K₂CO₃ (1.2 equivalents) is added. After 15 minutes a solution of naphthalene sulfonyl-chloride (or acyl-chloride for compound 6) (1.2 equivalents in dry THF) is added and the reaction is left stirring at room temperature. After 1-2 h the solvents are evaporated off under reduced pressure and the crudes are purified by flash chromatography (eluent DCM/MeOH 9/1-8/2) affording pure products.

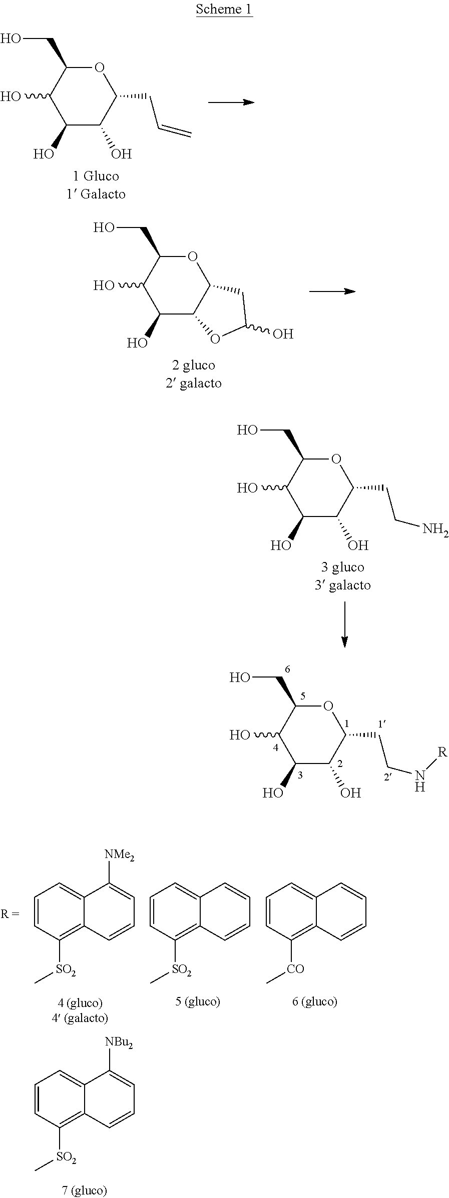

Scheme 2 reports the synthesis of compound 14. Compound 1 is fully benzylated with NaH (5 equivalents) and benzyl bromide (5 equivalents) in dry DMF affording compound 8. The latter is reacted with OsO₄ in the presence of NaIO₄ affording aldehyde 9 which is then reacted with [(ethoxycarbonyl)methylene]triphenylphosphorane to give the alpha-beta unsatured ester 10. Reduction with LiAlH₄ affords alcohol 11 that is converted to azide 12 through a Mitsunobu reaction with PPh₃ DIAD e (PhO)₂PON₃. Catalytic hydrogenation with H₂ and Pd(OH)₂ in MeOH/AcOEt directly gives amine 13, that is converted to compound 14 with the procedure already described.

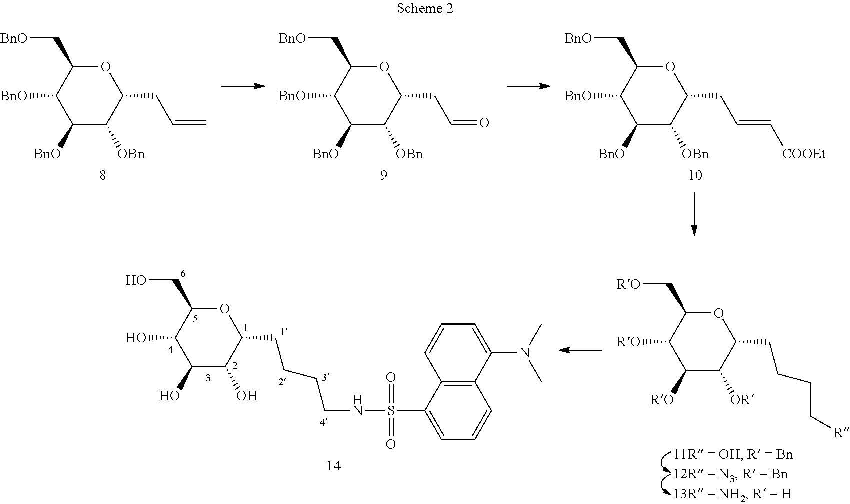

Scheme 3 reports the synthesis of compound 16 from amine 15 (La Ferla, 2005); a similar procedure already described for the other derivatives is exploited.

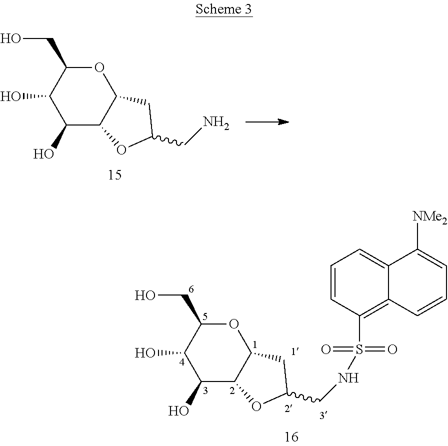

Stability Studies

Stability of compound 4 towards pH variations is evaluated through ¹H-NMR analysis. Two samples are prepared, one in acidic conditions and one in basic conditions; the first is prepared by dissolving 2.3 mg in 550 μl of D₂O and adjusting to pH 1.2 through DCl addition, while the second is prepared by dissolving 3 mg of compound 4 in 550 μl of D₂O and adjusting to pH 12.5 through NaOD addition. The two samples are kept at room temperature and ¹H-NMR spectra thereof are recorded at time 10 min, 4.5 h and 24 h. As evidenced by FIG. 1, no degradation of both samples is evidenced within 24 h.

### BRIEF DESCRIPTION OF THE FIGURES

FIG. 1. ¹H-NMR spectra of compound 4 at pH 12.5 and at pH 1.2.

FIG. 2A. LPS-induced IL-8 production by HT29 cells in the absence or presence of the indicated concentrations of compound (c) 4, 5, 6, 7, 4′, 14.

FIG. 2B. LPS (100 μg/ml)-induced IL-8 production by A549 human pneumocytes in the absence or presence of compound (Comp) 4 (5 μg/L).

FIG. 3. Serum levels of cytokine KC from mice treated with LPS alone or LPS with comp 4. Untr=untreated; LPS=Mice treated with LPS alone; comp 4+LPS=Mice treated with comp 4+LPS; comp 4=Mice treated with comp 4 alone.

FIG. 4. Protection afforded by comp 4 in LPS-induced shock in mice. Untr=untreated; LPSGalN=Mice treated with LPS/GalN alone; LPSGalN Comp 4=Mice treated with LPS/GalN+Comp 4=Mice treated with Comp 4 alone.

FIG. 5. TNF-α levels in mice treated with DSS alone or DSS+comp 4. Untr=Untreated; DSS=Mice treated with DSS alone; DSS comp 4=Mice treated with DSS and comp 4;=Mice treated comp 4 alone.

FIG. 6. Colon length in untreated mice or mice treated with DSS+comp 4. Untr=Untreated; DSS=Mice treated with DSS alone; DSS comp 4=Mice treated with DSS and comp 4;=Mice treated comp 4 alone.

FIG. 7. TNF-α content in the protein extract of the colon of untreated mice or mice treated with DSS+comp 4. Untr=Protein extract from colon of untreated mice; DSS=Protein extract from colon of mice treated with DSS alone; DSS comp 4=Protein extract from colon of mice treated with DSS+comp 4;=Protein extract from colon of mice treated with comp 4 alone.

FIG. 8. Serum levels of IL-4, IL-5 and IL-10 in untreated mice or mice treated with ovalbumin+comp 4. Untr=Untreated mice; comp 4=Mice treated with comp 4 alone; OVA=Mice treated with ovalbumin alone; comp 4+OVA=Mice treated with comp 4 and ovalbumin.

FIG. 9. BALF levels of IL-4 and IL-5 in untreated mice or mice treated with ovalbumin+compound 4. Untr=Untreated mice; comp 4=Mice treated with comp 4 alone; OVA=Mice treated with ovalbumin alone; comp 4+OVA=Mice treated with comp 4 and ovalbumin.

FIG. 10. Measurement of ROS in the cell culture medium of untreated enterocytes or enterocytes treated with doxorubicin+compound 4. Ctrl=Untreated (control) cells; Doxo=Cells treated with doxorubicin alone; Doxo comp 4=Cells treated with doxorubicin and compound 4.

FIG. 11. Serum levels of KC in untreated mice or mice submitted to acute treatment with doxorubicin or chronic treatment with doxorubicin and 5-fluorouracil. Untr=Untreated; Doxo 27 mg=Mice submitted to acute treatment with doxorubicin; Doxo 27 mg comp 4=Mice submitted to acute treatment with doxorubicin and concomitant p.o. administration of comp 4; Doxo cron=Mice submitted to chronic treatment with doxorubicin and 5-fluoruracil; Doxo cron comp 4=Mice submitted to chronic treatment with doxorubicin and 5-fluoruracil and concomitant p.o. administration of compound 4.

FIG. 12. Histology of the epithelium of the small intestine in mice after acute treatment with doxorubicin (DOXO) or acute treatment with doxorubicin and concomitant administration of compound 4 (comp 4+DOXO).

FIG. 13. Histology of the epithelium of the small intestine in mice after chronic treatment with doxorubicin and 5-fluoruracil (DOXO+FLUO) or chronic treatment with doxorubicin and 5-fluoruracil and concomitant administration of compound 4 (comp 4+DOXO+FLUO).

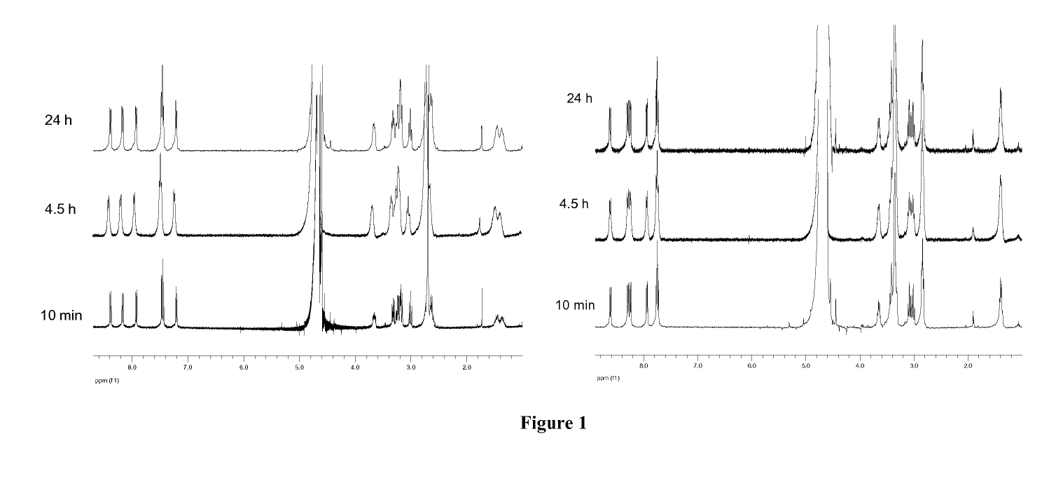

### EXPERIMENTAL SECTION

### Chemistry

General Remarks: All solvents were dried with molecular sieves, for at least 24 h prior to use. Thin layer chromatography (TLC) was performed on silica gel 60 F254 plates (Merck) with detection using UV light when possible, or by charring with a solution of concd. H₂SO4/EtOH/H₂O (5:45:45) or a solution of (NH₄)₆Mo₇O₂₄ (21 g), Ce(SO₄)₂ (1 g), concentred H₂SO₄ (31 mL) in water (500 mL) and heating at 150° C. Flash column chromatography was performed on silica gel 230-400 mesh (Merck). ¹H and 13C NMR spectra were recorded at 25° C. unless otherwise stated, with a Varian Mercury 400 MHz instrument. Chemical shift assignments, reported in ppm, are referenced to the corresponding solvent peaks. HRMS were recorded on a QSTAR elite LC/MS/MS system with a nanospray ion source, MS were recorded on an ESI QTRAP 2000 LC/MS/MS system. Optical rotations were measured at room temperature using an Atago Polax-2L polarimeter and are reported in units of 10⁻¹ deg·cm2·g⁻¹.

### 3,7-Anhydro-2-deoxy-α,β-D-glycero-L-galacto-octofuranose (1,4) (2)

A solution of allyl-C-glucoside 1 (1.5 g, 7.3 mmol) in MeOH/H₂O (100 ml) was cooled to −78° C. and saturated with ozone. After 75 min the pale blue solution was purged with oxygen and then with nitrogen until colourless. Then Me₂S (73 mmol, 10 equiv.) were added and the reaction mixture was warmed to room temperature and left stirring overnight. The solvent was then removed under reduced pressure affording crude compound 2a as a colourless oil. Crude compound 2, a mixture of hemiacetalic diastereoisomers, was used without purification.

3,7-Anhydro-2-deoxy-α,β-D-glycero-L-gulo-octofuranose (1,4) (2′): this compound was obtained with the same procedure used for the synthesis of 2. Starting material allyl-C-galactoside 1′ (2.0 g, 9.8 mmol) afforded crude compound 2′ as a colourless oil. The crude 2′, a mixture of hemiacetalic diastereoisomer containing also the two methyl acetal derivatives, was used without purification.

2-(α-D-Glucopyranosyl)ethanamine (3): Crude compound 2 (5.34 mmol, 1 equiv.) and anhydrous ammonium acetate (2.06 g, 5 equiv.) were suspended in dry toluene (10 ml) and evaporated to dryness. Under inert atmosphere dry MeOH (10 ml) and NaCNBH₃ (1.68 g, 5 equiv.) was added and the reaction was left stirring at r.t. After 1 h the solvent was removed under reduced pressure and the residue was purified using flash column chromatography over silica gel (AcOEt/MeOH/H₂O/AcOH, 6/2/1/1) to provide 3. ¹H NMR (400 MHz, D₂O) δ 3.95-3.85 (m, 1H, H(1)), 3.62 (dd, J=12.2, 1.98 Hz, 1H, H(6a)), 3.54-3.42 (m, 2H, H(2)(6b)), 3.38 (t, J=9.3 Hz, 1H, H(3)), 3.32-3.27 (m, 1H, H(5)), 3.12 (t, J=9.3 Hz, 1H, H(4)), 2.98-2.81 (m, 2H, H(2′a)(2′b)), 1.95-1.73 (m, 2H, H(1′a)(1′b)); 13C NMR (100 MHz, D₂O) δ 73.71, 73.10 72.98 70.59, 70.07 (5CH, C-1,2,3,4,5) 61.01 (CH₂, C-6), 36.93 (CH₂, C-2′), 22.23 (CH₂, C-1′).

2-(α-D-Galactopyranosyl)ethanamine (3′): Crude compound 2′ (1.43 mmol, 1 equiv.) was dissolved in dry MeOH (10 ml) and reacted with anhydrous ammonium acetate (5 equiv.) and NaCNBH₃ (5 equiv.) at room temperature. After 1 h the solvent was removed under reduced pressure and the residue was purified using flash column chromatography over silica gel (AcOEt/MeOH/H₂O/AcOH, 6/2/1/1) to provide 3′. ¹H NMR (400 MHz, CD₃OD) δ 4.04-3.98 (m, 1H), 3.85-3.80 (m, 2H), 3.68-3.52 (m, 4H), 3.05-2.95 (m, 2H), 2.01-1.85 (m, 2H); 13C NMR (100 MHz, CD₃OD) δ 76.14 (CH), 74.99 (CH), 72.31 (CH), 71.53 (CH), 70.46 (CH), 66.36 (CH₂), 63.86 (CH₂), 33.9 (CH₂).

Compounds 4, 5, 6, 7 and 4′ were obtained using the following procedure: amine 3/3′ was dissolved in dry MeOH (0.2 mmol/ml) and K₂CO₃ (1.2 equiv.) was added. After 15 min a solution of the sulfonyl/acyl chloride (1.2 equiv. in dry THF) was added and the reaction mixture was left stirring at room temperature. After 1-2 h the solvent was removed under reduced pressure and the crudes purified by flash chromatography (eluent DCM/MeOH 9/1-8/2).

### 5-(Dimethylamino)-N-[2′-(α-D-glucopyranosyl)ethyl]-1-naphthalenesulphonamide (4)

¹H NMR (400 MHz, D₂O) δ 8.50 (d, J=9.4 Hz, 1H, HAr), 8.28 (t, J=9.4 Hz, 2H, HAr), 7.74-7.65 (m, 2H, HAr), 7.42 (d, J=7.1 Hz, 1H, HAr), 3.88-3.76 (m, 1H, H(1)), 3.60-3.47 (m, 2H, H(2)(6a)), 3.29 (t, J=9.3 Hz, 1 H, H(3)), 3.19 (t, J=9.3 Hz, 1H, H(4)), 3.08-3.00 (m, 1H, H(5)), 2.95 (bt, 2H, H(2′a)(2′b)), 2.87 (s, 6H, N(CH₃)₂), 1.64-1.47 (m, 2H, H(1′a)(1′b)); 13C NMR (100 MHz, CD₃OD) δ 152.0, 135.6, 129.7 (CqAr); 130.0, 129.1, 128.0, 123.2, 119.3, 115.2 (6 CHAr); 73.97, 73.68, 73.38, 71.44, 70.92 (5CH, C-1,2,3,4,5); 61.82 (CH₂, C-6); 44.67 (CH₃N); 39.85 (CH₂, C-2′), 25.14 (CH₂, C-1′); HRMS calcd for [M+H]⁺ 441.1695: found 441.1435; [α]_{D}²⁰+135 (c=0.4, MeOH).

### N-[2′-(-(α-D-Glucopyranosyl)ethyl]-1-naphthalenesulfonamide (5)

¹H NMR (400 MHz, D₂O) δ 8.51 (d, J=8.5 Hz, 1H, HAr), 8.16 (t, J=8.8 Hz, 2H, HAr), 8.01 (d, J=8.1 Hz, 1H, HAr), 7.73 (t, J=7.6 Hz, 1H, HAr), 7.64 (bt, 1H, HAr), 7.56 (t, J=7.8 Hz, 1H, HAr), 3.87-3.76 (m, 1H, H(1)), 3.59 (dd, J=12.0, 1.8 Hz, 1H, H(6a)), 3.55-3.46 (m, 2H, H(2)(6b)), 3.29 (t, J=9.3 Hz, 1H, H(3)), 3.19 (t, J=9.3 Hz, 1H, H(4)), 3.08-3.00 (m, 1 H, H(5)), 2.91 (bt, 2H, H(2′a)(2′b)), 1.62-1.50 (m, 2H, H(1′a)(1′b)); 13C NMR (100 MHz, D₂O) δ 134.2, 132.7, 127.4 (CqAr); 135.1, 130.1, 129.6, 128.8, 127.3, 124.5, 123.5 (7 CHAr); 73.11, 73.03, 72.46, 70.65, 69.93 (5CH, C-1,2,3,4,5); 60.80 (CH₂, C-6); 39.12 (CH₂, C-2′), 23.83 (CH₂, C-1′); MS calcd for [M+H]⁺ 398.1; [M+Na]⁺ 420.1; found [M+H]⁺ 397.9; [M+Na]⁺ 420.1; [α]_{D}²⁰+43 (c=0.7, MeOH).

### N-[2′-(α-D-Glucopyranosyl)ethyl]-1-naphtalenecarboxyamide (6)

¹H NMR (400 MHz, D₂O) δ 7.87-7.79 (m, 2H, HAr), 7.78-7.73 (m, 1 H, HAr), 7.41-7.35 (m, 3H, HAr), 7.31 (t, J=7.6 Hz, 1H, HAr), 3.99-3.91 (m, 1H, H(1)), 3.57 (bd, 1H, H(6a)), 3.56-3.51 (m, 1H, H(2)), 3.49-3.31 (m, 5H, H(3)(5)(6b)(2′a)(2′b)), 3.18-3.13 (m, 1H, H(4)), 1.93-1.74 (m, 2H, H(1′a)(1′b)); 13C NMR (100 MHz, D₂O) δ 175.2 (C═O); 135.9, 135.7, 131.9 (CqAr); 133.4, 131.1, 130.0, 129.3, 128.0, 127.7, 127.1 (7 CHAr); 76.54, 75.88, 75.36, 73.57, 72.88 (5CH, C-1,2,3,4,5); 63.66 (CH₂, C-6); 39.26 (CH₂, C-2′), 26.03 (CH₂, C-1′); MS calcd for [M+H]⁺ 362.2; [M+Na]⁺ 384.1; found [M+H]⁺ 362.4; [M+Na]⁺ 384.5; [α]_{D}²⁰+55 (c=1.0, MeOH).

### 5-(Dibutylamino)-N-[2′-(α-D-glucopyranosyl)ethyl]-1-naphthalenesulfonamide (7)

¹H NMR (400 MHz, CD₃OD) δ 8.65 (d, J=8.5 Hz, 1H, HAr), 8.39 (d, J=8.5 Hz, 1H, HAr), 8.19 (d, J=7.2 Hz, 1H, HAr), 7.60 (dd, J=8.5, J=7.4 Hz, 1H, HAr), 7.55 (bdd, J=8.5, J=7.8 Hz, 1H, HAr), 7.37 (d, J=7.4 Hz, 1H, HAr), 3.94-3.82 (m, 1H, H(1)), 3.71 (dd, J=11.6, J=1.9 Hz, 1H, H(6a)), 3.59-3.47 (m, 2H, H(2)(6b)), 3.41 (t, J=8.9 Hz, 1H, H(3)), 3.35-3.30 (m, 1H, H(5)), 3.20-3.15 (m, 1H, H(4)), 3.13 (bt, 4H, N(CH₂CH₂CH₂CH₃)₂)), 3.05-2.84 (m, 2H, H(2′a)(2′b)), 1.88-1.75 (m, 2H, H(1′a)(1′b)), 1.52-1.40 (m, 4H, N(CH₂CH₂CH₂CH₃)₂)), 1.35-1.25 (m, 4H, N(CH₂CH₂CH₂CH₃)₂)), 0.85 (t, J=7.4 Hz, 6H, N(CH₂CH₂CH₂CH₃)₂)), 13C NMR (100 MHz, CD₃OD) δ 153.5, 136.3, 133.7 (CqAr); 133.9, 133.3, 131.5, 127.1, 123.9, 123.3 (6 CHAr); 77.93, 77.64, 77.40, 75.43, 74.94 (5CH, C-1,2,3,4,5); 65.81 (CH₂, C-6); 58.25 (N(CH₂CH₂CH₂CH₃)₂)); 43.77 (CH₂, C-2′); 33.24 (N(CH₂CH₂CH₂CH₃)₂)); 29.21 (N(CH₂CH₂CH₂CH₃)₂)); 24.29 (CH₂, C-1′); 17.06 (N(CH₂CH₂CH₂CH₃)₂)); MS calcd for [M+H]⁺ 525.3; found [M+H]⁺ 525.1; [α]_{D}²⁰+28 (c=0.6, MeOH).

### 5-(Dimethylamino)-N-[2′-(α-D-galactopyranosyl)ethyl]-1-naphthalenesulfonamide (4′)

¹H NMR (400 MHz, D₂O) δ 8.32 (d, J=8.5 Hz, 1H, HAr), 8.16 (t, J=7.3 Hz, 2H, HAr), 7.52 (q, J=8.4 Hz, 2H, HAr), 7.25 (d, J=7.4 Hz, 1H, HAr), 3.73-3.64 (m, 1H, H(1)), 3.61 (m, 2H, H(2)(4)), 3.39-3.30 (m, 2H, H(6a)(6b)), 3.16 (dd, J=9.8, J=3.5 Hz, 1H, H(3)), 3.12-3.06 (m, 1H, H(5)), 2.79 (bt, 2H, H(2′a)(2′b)), 2.69 (s, 6H, N(CH₃)₂), 1.36-1.26 (m, 2H, H(1′a)(1′b)); 13C NMR (100 MHz, CD₃OD) δ 153.2, 137.0, 131.2, 131.0 (CqAr); 131.2, 130.4, 129.2, 124.4, 120.6, 116.5 (6 CHAr); 74.31, 71.94, 71.94, 70.09, 70.09 (5CH, C-1,2,3,4,5); 62.16 (CH₂, C-6); 45.86 (CH₃N); 41.31 (CH₂, C-2′), 26.83 (CH₂, C-1′); MS calcd for [M+H]⁺ 441.2; [M+Na]+463.2; found [M+H]⁺ 441.3; 463.1; [α]^{D}₂₀+33 (c=0.7, MeOH).

(2,3,4,6-Tetra-O-benzyl-α-D-glucopyranosyl)acetaldehyde (9): (2,3,4,6-Tetra-O-Benzyl-α-D-glucopyranosyl)propene 8 (400 mg, 0.71 mmol) was dissolved in BuOH/Acetone/H₂O 1/1/1 (10 ml) and NaIO₄ (758 mg, 5 equiv.) was added. After 20 min OsO₄ (solution 0.016 M in tBuOH, 0.05 equiv.) was added and the reaction was stirred at room temperature. After 1.5 h the reaction was concentrated then was diluted in AcOEt, washed with H₂O. The organic layer was dried (Na₂SO₄), filtered and the filtrate concentrated under vacuum. The resulting crude aldehyde was used for the following reaction.

Ethyl 4-(2′,3′,4′,6′-tetra-O-benzyl-α-D-glucopyranosyl)but-2-enoate (10): crude aldehyde 9 was dissolved in toluene (20 ml) and (triphenyl-λ⁵-phosphanylidene)-acetic acid ethyl ester (1.5 equiv) was added and the reaction was left stirring at r.t. After 2 h the reaction was concentrated and the crude purified by flash chromatography (eluent Petroleum ether/AcOEt 9/1-8/2) to afford pure 10. ¹H NMR (400 MHz, CDCl₃) δ 7.40-7.08 (m, 20H, HAr), 6.96 (dt, J=15.1, J=7.5 Hz, 1H, CH═CHCOOEt), 5.91 (d, J=15.1 Hz, 1H, CH═CHCOOEt), 4.93 (d, J=10.9 Hz, 1H, CHPh), 4.81 (bd, J=11.1 Hz, 2H, 2CHPh), 4.72 (d, J=11.6 Hz, 1H, CHPh), 4.63 (d, J=12.0 Hz, 1H, CHPh), 4.61 (d, J=11.5 Hz, 1H, CHPh), 4.50-4.42 (m, 2H, 2CHPh), 4.22-4.12 (m, 3H, COOCH₂CH₃, H(1)), 3.81-3.52 (m, 6H, H(2)(3)(4)(5)(6a)(6b)), 2.71-2.45 (m, 2H, H(1′a)(1′b)), 1.28 (t, J=7.1 Hz, 3H, COOCH₂CH₃); 13C NMR (100 MHz, CDCl₃) δ 166.6 (C═O); 145.4 (CH═); 138.8, 138.3 (CqAr); 128.7-127.9 (CHAr); 123.7 (CH═); 82.49, 79.92, 78.03, 73.48, 71.67 (5CH, C-1,2,3,4,5); 75.75, 75.36, 73.75, 73.62 (4CH₂Ph); 71.67 (CH₂, C-6); 60.50 (COOCH₂CH₃); 28.66 (CH₂, C-1′); 14.51 (COOCH₂CH₃); MS calcd for [M+H]⁺ 637.3; [M+Na]⁺ 659.3; found [M+H]⁺ 637.4; [M+Na]⁺ 659.3; [α]_{D}²⁰+46 (c=1.3, CHCl₃).

4-(2′,3′,4′,6′-Tetra-O-benzyl-α-D-glucopyranosyl)butan-1-ol (11): compound 10 (250 mg, 0.39 mmol) was dissolved in dry THF (3 ml) under inert atmosphere and the solution cooled to 0° C.; LiAlH₄ (1.6 ml of 1M solution in THF) was then added and the reaction left to warm to room temperature. After 2 h the reaction was cooled to 0° C. and H₂O (61 μl), NaOH 4M (75 μl), H₂O (190 μl), and Na₂SO₄ (441 mg) were added in order. After 2 h the precipitate was filtered and the filtrate evaporated under reduced pressure. The crude purified by flash chromatography (eluent Petroleum ether/AcOEt 7/3) to afford pure 11. ¹H NMR (400 MHz, CDCl₃) δ 7.40-7.08 (m, 20H, HAr), 4.96 (d, J=10.9 Hz, 1H, CHPh), 4.84 (d, J=10.6 Hz, 1H, CHPh), 4.82 (d, J=10.9 Hz, 1H, CHPh), 4.71 (d, J=11.6 Hz, 1H, CHPh), 4.63 (bd, 2H, 2CHPh), 4.54-4.45 (m, 2H, 2CHPh), 4.09-4.00 (m, 1H, H(1)), 3.86-3.72 (m, 2H, H(2)(3)), 3.70-3.55 (m, 6H H(4)(5)(6a)(6b), —CH₂CH₂OH), 1.84-1.47 (m, 5H, —CH₂CHCH₂CH₂OH), 1.44-1.31 (m, 1H, —CH₂CHCH₂CH₂OH); 13C NMR (100 MHz, CDCl₃) δ 139.0, 138.5, 138.4, 138.2 (CqAr); 128.7-127.9 (CHAr); 82.82, 80.51, 78.48, 74.17, 71.22 (5CH, C-1,2,3,4,5); 75.79, 75.37, 73.75, 73.37 (4CH₂Ph); 69.30 (CH₂, C-6); 63.00 (CH₂OH); 32.64, 24.61, 21.75 (—CH₂CH₂CH₂CH₂OH); MS calcd for [M+H]⁺ 597.3; [M+Na]⁺ 619.3; found [M+H]⁺ 697.2; [M+Na]⁺ 619.4; [α]_{D}²⁰+89 (c=0.3, CHCl₃).

1-Azido-4-(2′,3′,4′,6′-tetra-O-benzyl-α-D-glucopyranosyl)butane (12): compound 11 (90 mg, 0.15 mmol) was dissolved in dry THF (1 ml) under inert atmosphere and Ph₃P (119 mg, 3 equiv.) was added. The solution was cooled to 0° C. and diisopropylazodicarboxylate (DIAD) (88 μl, 3 equiv.) was added drop wise in 10 min. Then (PhO)₂P(O)N₃ (104 μl, 3.2 equiv.) was added and the reaction left warming to r.t. After 2 h the solvent was removed under reduced pressure and the crude purified by flash chromatography (eluent Petroleum ether/AcOEt 9/1) to afford pure 12 (72 mg, 77%). ¹H NMR (400 MHz, CDCl₃) δ 7.40-7.08 (m, 20H, HAr), 4.98 (d, J=10.9 Hz, 1H, CHPh), 4.86 (d, J=10.6 Hz, 1H, CHPh), 4.85 (d, J=10.9 Hz, 1H, CHPh), 4.75 (d, J=11.7 Hz, 1H, CHPh), 4.66 (d, J=12.1 Hz, 1H, CHPh), 4.65 (d, J=11.6 Hz, 1H, CHPh), 4.52 (bt, 2H, 2CHPh), 4.10-3.99 (m, 1H, H(1)), 3.86-3.55 (m, 6H, H(2)(3)(4)(5)(6a)(6b), 3.28 (t, J=6.73Hz, 3H, —CH₂CH₂N₃), 1.87-1.48 (m, 5H, —CH₂CHCH₂CH₂N₃), 1.47-1.34 (m, 1H, —CH₂CHCH₂CH₂N₃); 13C NMR (100 MHz, CDCl₃) δ 139.0, 138.5, 138.4, 138.3 (CqAr); 128.7-127.9 (CHAr); 82.80, 80.48, 78.42, 74.11, 71.32 (5CH, C-1,2,3,4,5); 75.05, 75.38, 73.77, 73.50 (4CH₂Ph); 69.27 (CH₂, C-6); 51.59 (CH₂N₃); 28.89, 24.46, 22.87 (—CH₂CH₂CH₂CH₂N₃); MS calcd for [M+H]⁺ 622.3; [M+Na]⁺ 644.3; [M+K]⁺ 660.3; found [M+H]⁺ 622.4; [M+Na]⁺ 644.4; [M+K]⁺ 660.3; [α]_{D}²⁰+20 (c=0.2, CHCl₃).

4-(α-D-Glucopyranosyl)butanamine (13): compound 12 (60 mg, 0.097 mmol) was dissolved in AcOEt/MeOH 1/1 (3 ml) and AcOH (100 μl), then a catalytic amount Pd(OH)₂ was added and the reaction was stirred under H₂ atmosphere over night. The reaction was filtered through a celite pad and concentrated under reduced pressure affording amine 13. ¹H NMR (400 MHz, D₂O) δ 3.86-3.76 (m, 1H, H(1)), 3.72 (dd, J=11.7, J=2.3 Hz, 1H, H(6a)), 3.55-3.47 (m, 2H, H(2)(6b)), 3.44 (dd, J=9.4, J=8.5 Hz, 1H, H(3)), 3.34-3.27 (m, 1H, H(5)), 3.12 (dd, J=9.4, J=8.6 Hz, 1H, H(4)), 2.86 (t, J=7.5 Hz, 2H, —CH₂CH₂NH₂), 1.73-1.40 (m, 5H, —CH₂CHCH₂CH₂NH₂), 1.38-1.24 (m, 1H, —CH₂CHCH₂CH₂NH₂); 13C NMR (100 MHz, D₂O) δ 79.52, 77.98, 77.30, 75.76, 75.21 (5CH, C-1,2,3,4,5) 66.05 (CH₂, C-6), 43.54 (CH₂NH₂); 31.02, 27.72, 26.24 (—CH₂CH₂CH₂CH₂NH₂); MS calcd for [M+H]⁺ 236.2; found [M+H]⁺ 236.2; [α]_{D}²⁰+16 (c=0.2, MeOH).

5-(Dimethylamino)-N-[4′-(α-D-glucopyranosyl)butyl]-1-naphthalenesulfonamide (14): amine 13 was dissolved in dry MeOH (0.7 ml) and K₂CO₃ (15 mg, 1.7 equiv.) was added. After 15 min a solution of the dansyl chloride (40 mg, 1.7 equiv. in dry THF 0.7 ml) was added and the reaction mixture was left stirring at room temperature. After 1-2 h the solvent was removed under reduced pressure and the crudes purified by flash chromatography (eluent DCM/MeOH 9/1) to afford pure 14. ¹H NMR (400 MHz, CD₃OD, 37° C.) δ 8.54 (d, J=8.5 Hz, 1H, HAr), 8.36 (d, J=8.6 Hz, 1H, HAr), 8.18 (d, J=7.3 Hz, 1H, HAr), 7.61-7.49 (m, 2H, HAr), 7.26 (d, J=7.54 Hz, 1H, HAr), 3.77-3.67 (m, 2H, H(1)(6a)), 3.59 (dd, J=11.7, J=5.5 Hz, 1H, H(6b)), 3.51 (dd, J=9.3, J=5.7 Hz, 1H, H(2)), 3.44 (t, J=8.8 Hz, 1H, H(3)), 3.36-3.25 (m, 1H, H(5)), 3.20 (t, J=8.8 Hz, 1H, H(4)), 2.92-2.79 (m, 8H, H(2′a)(2′b) N(CH₃)₂), 1.58-1.22 (m, 6H, CH₂CH₂CH₂CH₂NHR); 13C NMR (100 MHz, CD₃OD, 37° C.) δ 156.0, 140.0, 134.0, 133.8 (CqAr); 133.8, 132.9, 131.8, 127.0, 123.4, 119.2 (6 CHAr); 79.72, 78.02, 77.11, 75.84, 75.19 (5CH, C-1,2,3,4,5); 65.96 (CH₂, C-6); 48.57 (CH₃N); 46.68 (CH₂NHR); 33.22, 27.74, 26.43 (—CH₂CH₂CH₂CH₂NHR); MS calcd for [M+H]⁺ 469.2; found [M+H]⁺ 469.4; [α]_{D}²⁰+100 (c=0.3, MeOH).

Compound 16: Amine 15 40 mg (0.18 mmol) was dissolved in dry MeOH (3 ml) and K₂CO₃ (23 mg, 1.2 equiv.) was added. After 20 min dansyl chloride (58 mg, 1.2 equiv.) was added and the reaction was stirred at room temperature. After 1 h the solvent was removed under reduced pressure and the crude purified by flash chromatography (eluent AcOEt/MeOH 9/1) to afford compound 16 as mixture of diastereoisomers. Major isomer: ¹H NMR (400 MHz, CD₃OD) δ ppm 8.56 (d, J=8.4 Hz, 1H, HAr), 8.35 (d, J=8.72 Hz, 1H, HAr), 8.20 (d, J=7.02 Hz, 1H, HAr), 7.64-7.51 (m, 2H, HAr), 7.27 (d, J=7.6 Hz, 1H, HAr), 4.48-4.36 (m, 1H, H(1)), 3.95-3.83 (m, 1H, H(2′)), 3.77-3.33 (m, 6H, H(2)(3)(4)(5)(6a)(6b)), 3.09-2.93 (m, 2H, H(3′a)(3′b)), 2.92-2.81 (m, 6H, N(CH₃)₂), 2.05-1.94 (m, 1H, H(1′a)), 1.77-1.64 (m, 1H, H(1′b)); 13C NMR (100 MHz, CD₃OD, 37° C.) δ 155.9, 139.7, 133.9, 133.7 (CqAr); 134.0, 132.9, 132.0, 127.1, 123.3, 119.2 (6 CHAr); 86.74, 81.69, 81.04, 78.31, 77.74, 72.29 (6CH, C-1,2,3,4,5,2′); 65.10 (CH₂, C-6); 50.80 (CH₂, C-3′); 48.60 (CH₃N); 37.66 (CH₂, C-1′); MS calcd for [M+1-1]⁺ 453.2; found [M+H]⁺ 453.4.

Pharmacology

Anti-Inflammatory Activity of the Compounds

Effect on Epithelial Cells (Enterocytes and Pneumocytes) Stimulated with LPS

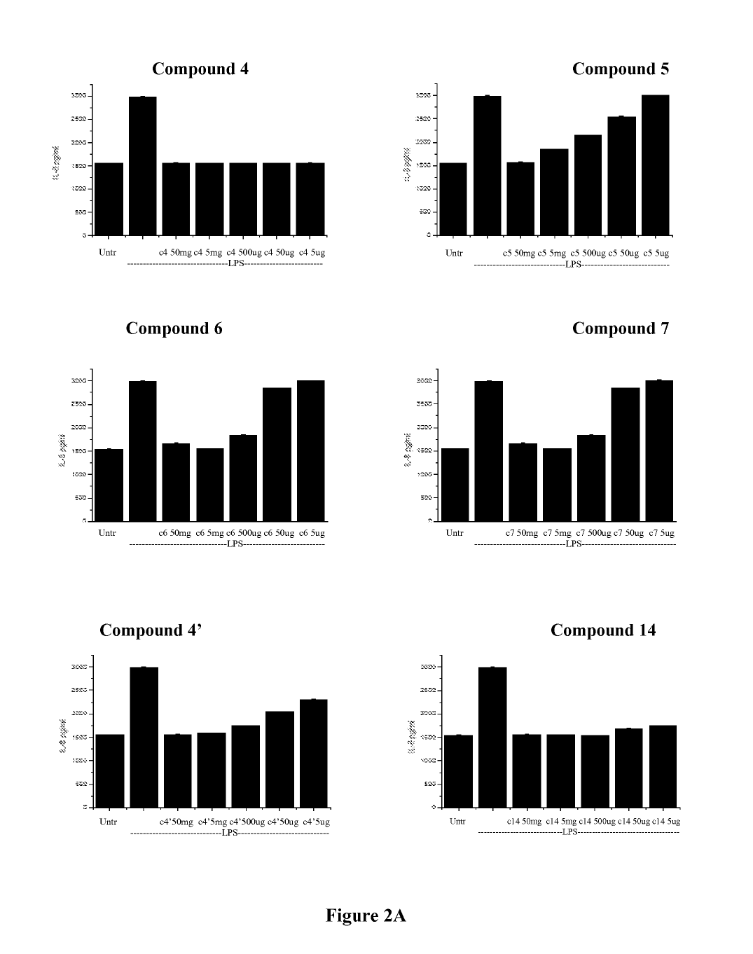

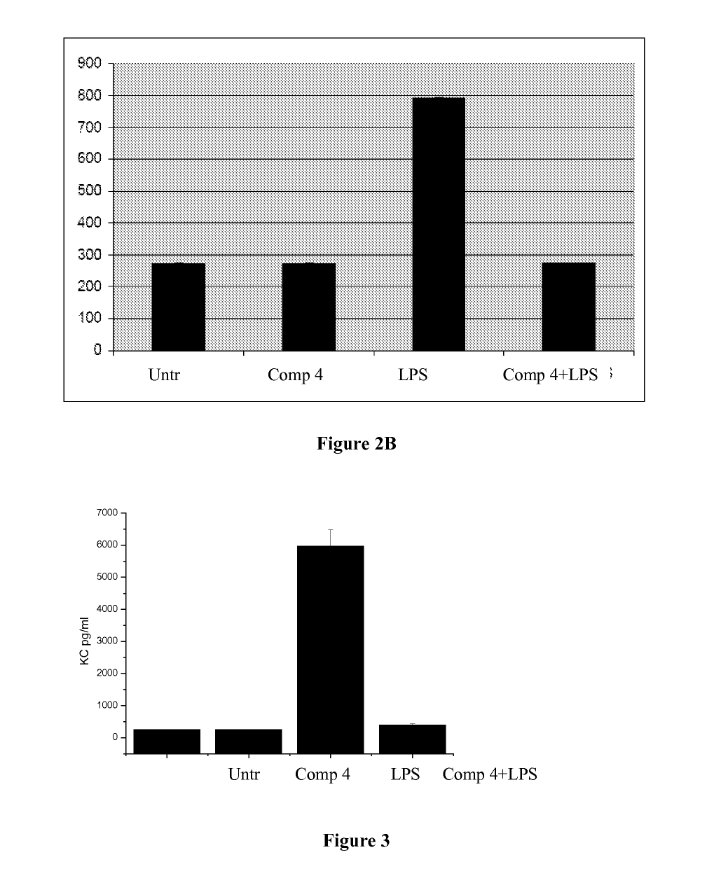

The effect of the compounds on the release of IL-8 or KC was tested upon stimulation of intestinal epithelial cells (IEC) with LPS from Salmonella enterica abortus equi. The human IEC line HT29 was used for these experiments. Cells were cultured for 18 h in medium alone or in medium additioned with different concentrations of compounds 4, 5, 6, 7, 4′ and 14 (from 50 mg/L to 5 μg/L). Then, cells were stimulated for 6 h with LPS (1 μg/ml). As determined by ELISA, the addition of the compounds significantly inhibited the production of IL-8 induced by LPS (FIG. 2A). Similar experiments were performed on a human pneumocyte type II cell line (A549) using compound 4 and LPS from Pseudomonas aeruginosa. As before, also in this case LPS-induced IL-8 production was significantly inhibited (FIG. 2B).

Treatment of Diarrhoea

We tested whether oral pretreatment of mice with the compound 4 interferes with the increase in KC serum levels induced by LPS administration to mice. The dosage of compound 4 chosen for all in vivo experiments was 25 μg/kg. Mice (10/group) were treated orally with compound 4 (25 μg/kg), followed by oral treatment with LPS (50 mg/kg). Serum samples were collected after 4 h, and KC levels were evaluated by ELISA. Mice treated with LPS showed a significant increase in serum KC concentrations, whereas the levels of this chemokine in sera of mice pretreated with compound 4 were comparable to those of untreated mice (FIG. 3).

Protection Afforded by Compound 4 in LPS-Induced Shock in Mice

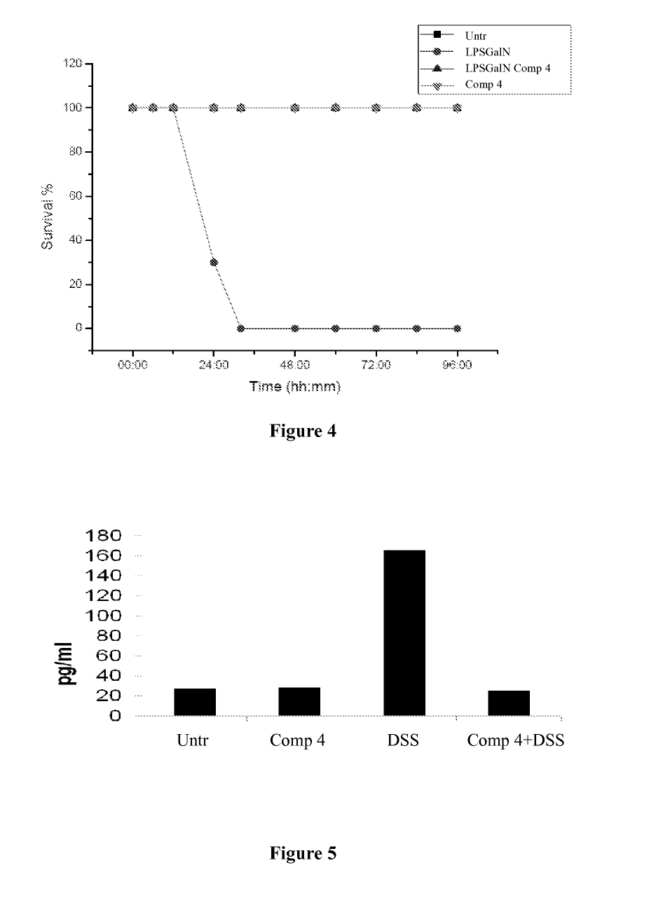

Compound 4 was tested in LPS/galactosamine (GalN)-induced shock in mice. LPS shock was induced in 2-months old FVB mice through intraperitoneal (i.p). injection of 5 μg LPS/mouse (LPS from Salmonella enterica abortus equi) and 40 mg/mouse GalN (LPS/GalN). 25 μg/kg (0.5 μg/mouse) of p.o. administered compound 4 were sufficient to afford full protection against LPS/GalN-induced shock (FIG. 4).

Treatment of Acute Colitis with Compound 4

In mice induced for acute Crohn's disease, modifications of epithelial permeability of the intestine were determined by measuring intestinal transmembrane resistance using the Ussing chamber. For this purpose, 2-months old FVB mice were administered dextran sulfate sodium (DSS) 2% in the drinking water for 7 days (Wirtz et al, 2007). Results of this experiment are shown in Table 1. As can be seen resistance in mice treated with DSS+4 was significantly increased as compared to mice treated with DSS alone. This suggests that compound 4 but at much lower doses, leads to recovery of normal epithelial permeability in DSS-treated mice. Eventually, the serum levels of the inflammatory cytokine TNF-α were measured in mice treated with DSS alone or with DSS+4. FIG. 5 shows the results. The treatment with compound 4 strongly inhibited the increase induced by DSS 2% and the subsequent acute colitis.

TABLE 1
Intestinal transmembrane resistance in mice treated with DSS +
compound 4 for induction of acute Crohn-like disease.

| | | | | DSS + |
|---|---|---|---|---|
| | Untreated | compound 4 alone | DSS alone | compound 4 |
| Resistance in | 41 | 40.8 | 20.8 | 49 |
| Ω × cm² | | | | |

Treatment of Crohn's Disease with Compound 4

Chronic Crohn-like disease was induced in mice by administration of DSS 2% for 4 cycles, with each cycle consisting in exposition to DSS 2% for 7 days, followed by 14 days of regular water. In mice that were administered also compound 4, last exposition of DSS was followed by p.o. administration of compound 4 at 25 μg/kg, 4 times/week for 3 weeks. Results of this experiment are shown in Table 2. As can be seen, resistance in mice treated with DSS+4 is significantly increased as compared to mice treated with DSS alone.

TABLE 2
Intestinal transmembrane resistance in mice treated with DSS +
compound 4 for induction of chronic Crohn-like disease.

| | | | | DSS + |
|---|---|---|---|---|
| | Untreated | compound 4 alone | DSS alone | compound 4 |
| Resistance in | 48.3 | 45.1 | 18.8 | 47 |
| Ω × cm² | | | | |

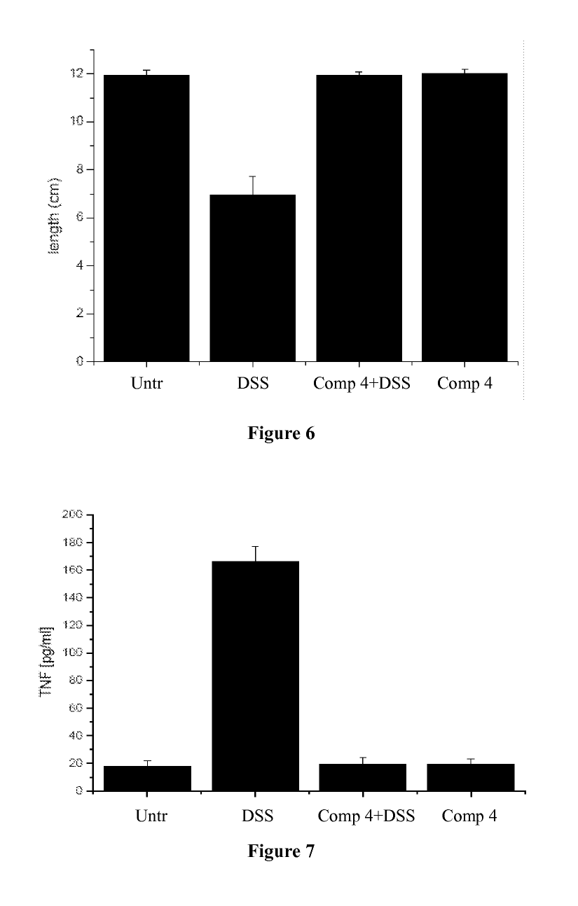

One hallmark of the chronic Crohn's disease model is the shortening of the colon. Therefore, the length of the colon of mice treated with DSS+4 was measured in order to see if compound 4 had an impact also on this disease parameter. It was found that the mean length of the colon of mice treated with DSS 2% alone was 7 cm, while that of mice treated with DSS 2%+4 was 11.55 cm, similar to that of untreated mice (FIG. 6).

Another parameter that was measured in this setting was the TNF-α content in the protein extract of the colon of mice. The results of this experiment are shown in FIG. 7. DSS 2% alone led to a dramatic increase of the TNF-α levels in the protein extract, while co-treatment with compound 4 led to normalization of these levels.

Treatment of Asthma with Compound 4.

Compound 4 was tested in a mouse model of asthma where glucose had already been shown therapeutic efficacy. For this purpose, 10 male C57B1/6 mice were immunized intraperitoneally with 100 μg of ovalbumin (grade V) suspended in 500 μg Al(OH)₃. Another group of 10 animals was treated the same way and, in addition, also with compound 4 (25 μg/kg) administered orally. One week after the first immunization, the same treatments were repeated. Two weeks after the first immunization, the first group of animals was challenged for 25 min with an aerosol of 5% (wt/vol) ovalbumin every day, for 5 consecutive days, while the other group received aerosolized ovalbumin plus compound 4 (25 μg/kg). The results showed that, indeed, compound 4 afforded protection also in this inflammatory disease model. Such protection was demonstrated at different levels.

Thus, analysis of the bronchoalveolar lavage fluid (BALF) showed a marked increase of eosinophils in the lungs of ovalbumin-treated mice (4068 cells/ml), while animals treated with compound 4 and ovalbumin showed a limited number of eosinophils in the alveolar spaces (127 cells/ml).

A hallmark of inflammatory damage to the lung is the development of high-permeability edema, characterized by high protein content in the edema fluid. Indeed, in the lungs of ovalbumin-exposed mice the protein concentration in the BALF was markedly high (0.729 mg/ml), witnessing an important damage to the lungs of these animals. On the other hand, the protein concentration in the BALF of ovalbumin+4-treated animals was similar to that of untreated animals (0.394 mg/ml).

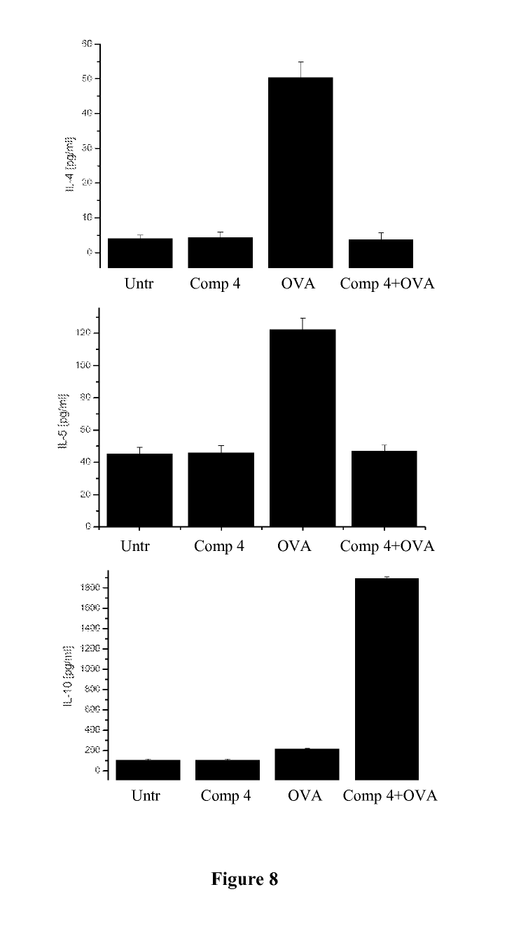

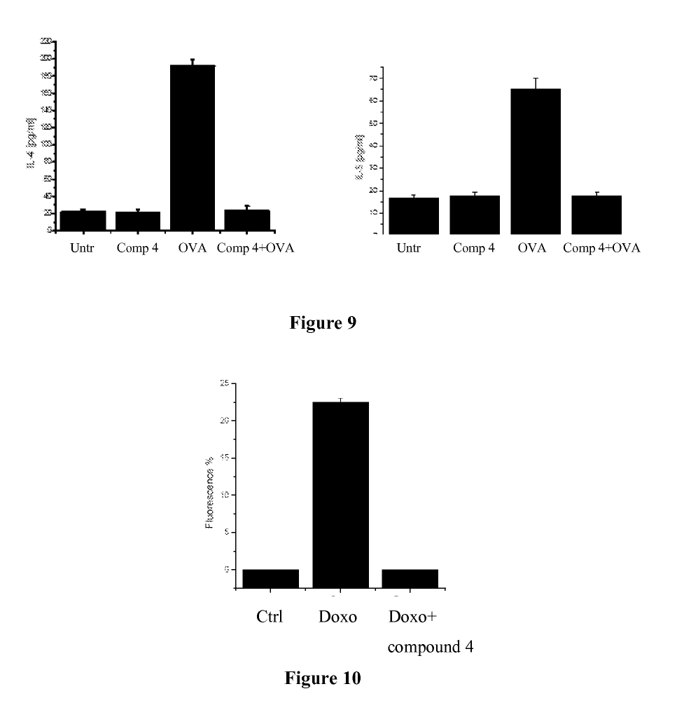

Eventually, also the cytokine levels in the sera and BALFs of ovalbumin- or ovalbumin+compound 4-treated animals were measured. The levels of the cytokines IL-5 and IL-4, which are pivotal in the pathogenesis of asthmatic disease, were elevated in both sera and BALF from ovalbumin-treated mice, but normal in ovalbumin+4-treated animals. On the other hand, IL-10 was greatly increased in the sera of mice treated with ovalbumin+4, while it remained at low levels in ovalbumin-treated mice (FIGS. 8 and 9).

The same experiments are performed with oral administration of compound 4 during challenging for 25 min with an aerosol of 5% (wt/vol) ovalbumin; the data demonstrated that the oral challenge protects the ovalbumin inflammation analogously to the aerosol challenge, too (data not shown).

Altogether, the present results suggest that asthma is another inflammatory disease, in addition to Crohn's disease, that can be therapeutically addressed with the highly potent glucose analogue 4.

Compound 4 protects enterocytes from chemotherapy-induced injury.

Compound 4 has shown protective efficacy also in an in vitro model of chemotherapy-induced injury of enterocytes. In this model, a monolayer of Caco-2 enterocytes was treated with 3 μM doxorubicin alone or doxorubicin and 4. Compound 4 showed a clear protective activity against doxorubicin-induced injury (FIG. 10). Thus, evaluation of the amount of reactive oxygen species (ROS) in the cell culture medium showed a dramatic increase of ROS in the medium of doxorubicin-treated cells and a complete inhibition in cells treated with doxorubicin+4.

On the basis of these in vitro results, compound 4 was also tested in vivo: first, in an acute model of doxorubicin-induced enterocyte injury, then in a chronic model of doxorubicin- and 5-fluoruacil-induced enterocyte injury.

For the first model, doxorubicin was administered to mice by intraperitoneal injection (27 mg/kg). Some of the mice were administered at the same time compound 4 (25 μg/kg orally). Recovery of the blood for the determination of circulating KC levels and subsequent sacrifice of the animals were performed at 72 h after treatment.

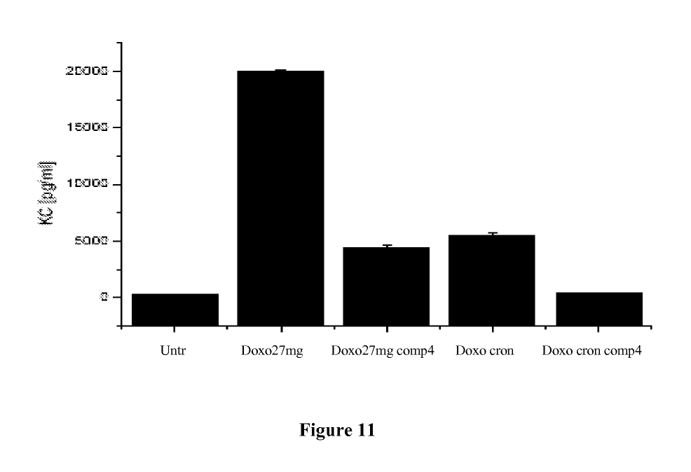

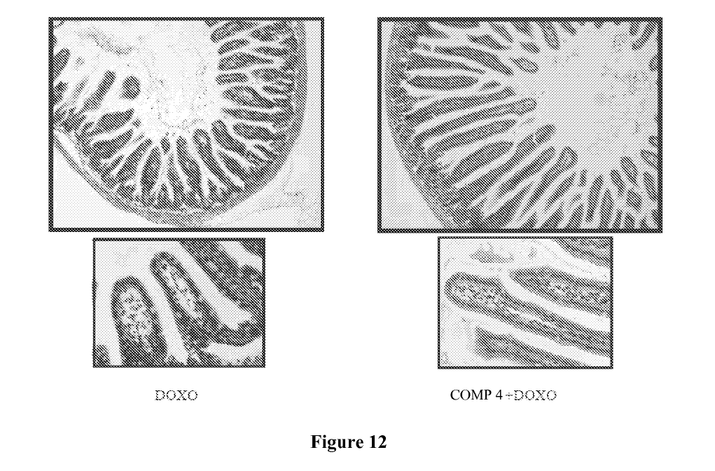

As can be seen from FIG. 11, circulating KC levels greatly increased in mice submitted to acute treatment with doxorubicin (second column). These levels were strongly reduced in mice that were administered at the same time compound 4 (third column). Histological examination of the epithelium of the small intestine (FIG. 12) shows that mice submitted to acute treatment with doxorubicin have shortened villi and damages to the connective tissue. A normal morphology is seen in mice that were treated at the same time with compound 4.

For the chronic model of enterocyte injury, doxorubicin was administered 1×/week for three weeks (7 mg/kg for the first two administrations, 100 mg/kg for the last administration). At the same times, mice were administered also 5-fluoruracil at 100 mg/kg. Administrations of the chemotherapeutics were performed intraperitoneally. Some of the mice were treated at the same times with compound 4 (25 μg/kg, orally). Recovery of the blood for the determination of circulating KC levels and subsequent sacrifice of the animals were performed at 72 h after the last treatment.

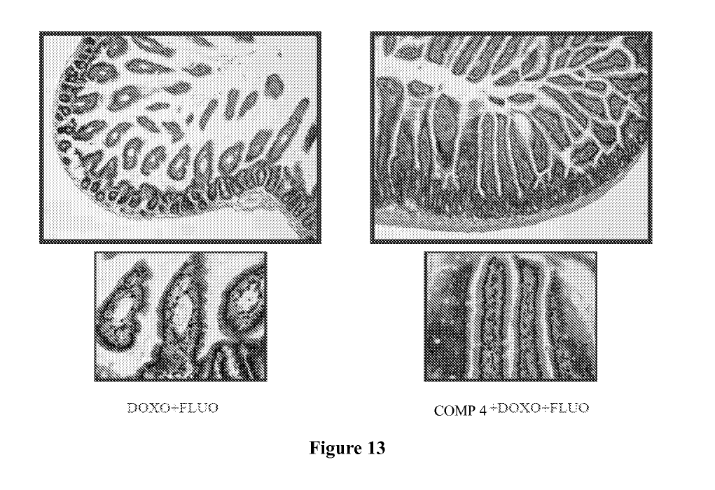

The results of these experiments are shown in FIG. 11 (fourth and fifth column) and in FIG. 13. As can be seen from FIG. 11, circulating KC levels increased in mice submitted to chronic treatment with doxorubicin and 5-fluoruracil (fourth column). The increase is less striking than that observed for acute treatment. In this case administration of compound 4 brought the levels back to those observed in untreated mice.

Histological examination of these mice gave results similar to those after acute treatment with doxorubicin (FIG. 13). Thus, following treatment with the chemotherapeutics alone, the small intestine shows very short villi and damages to the connective tissue. A completely normal morphology, on the other hand, is seen in mice that had been co-treated with compound 4.

Compound 4 reduces glycemia in mice after glucose challenge.

All evidence gathered with compound 4 suggests that this compound acts as a non-metabolizable glucose agonist at SGLT-1. As such, it was argued that it might act as a functional antagonist of SGLT-1-mediated intestinal glucose absorption, and used for the control of hyperglycemia in type II diabetes. This possibility was tested in the following experiments.

In a first set of experiments mice were orally challenged with 2.5 g/kg of glucose with or without concomitant administration of compound 4 (250 μg/kg) and glycemia was measured after 1 h. As can be seen from Table 3, co-administration of compound 4 completely inhibited increase of glycemia.

TABLE 3
Glycemia in mice after oral glucose challenge with or without
concomitant administration of compound 4.

| Plasma from | Glycemia levels |
|---|---|
| Control mice. | 127 mg/dl |
| Mice challenged with 2.5 g/kg of | 285 mg/dl |
| glucose. | |
| Mice challenged with 250 μg/kg of | 133 mg/dl |
| compound 4. | |
| Mice challenged with 2.5 g/kg of | 150 mg/dl |
| glucose + 250 μg/kg of compound 4. | |

These results show that compound 4 inhibits glucose adsorption, thereby reducing glycemia after glucose challenge.

### REFERENCES

Bennek J A, Gray G A. An efficient synthesis of anhydroalditols and allylic-glycosides J. Org. Chem. 1987, 52, 892-897.
Braat H, Rottiers P, Hommes D W, Steidler L. A phase I trial with transgenic bacteria expressing interleukin-10 in Crohn's disease. Clin. Gastroenterol Hepatol. 2006, 4(6): 754-9.
Chadda K, Annane D. The use of corticosteroids in severe sepsis and acute respiratory distress syndrome. Ann Med. 2002; 34(7-8):582-9.
Corry D B, Kheradmand F. Biology and therapeutic potential of the interleukin-4/interleukin-13 signaling pathway in asthma. Am J Respir Med. 2002; 1(3):185-93.
Diez-Sampedro A., Wright E. M., Hirayama B. A, 2001 J. Biol. Chem. 276, 49188-49194
Dziarski R, Gupta D. Role of MD-2 in TLR2- and TLR4-mediated recognition of Gram-negative and Gram-positive bacteria and activation of chemokine genes. J Endotoxin Res. 2000; 6(5):401-5.
Eckhardt, M.; Himmelsbach, F.; Eickelmann, P.; Thomas, L.; Barsoumian, E. L. Preparation of aryl C-glycosides and their inhibiting effect on the sodium-dependent glucose co-transporter SGLT. (Boehringer Ingelheim). US 2006035841.
Fujikura, H.; Fushimi, N.; Isaji, M. Preparation of glucopyranoside compounds containing phenol moiety as SGLT inhibitors. (Kissei Pharmaceutical). WO 2005-JP6702 20050330.
Fushimi, N.; Fujikura, H.; Isaji, M. Preparation of glucopyranose compounds containing fused heterocycle moiety as SGLT inhibitors. (Kissei Pharmaceutical) WO 2005085265.
Fushimi, N.; Teranishi, H.; Shimizu, K.; Yonekubo, S.; Ito, F; Isaji, M. Preparation of nitrogenous fused-ring glycoside derivatives as inhibitors of human sodium-dependent glucose transporter (SGLT). (Kissei Pharmaceutical Co., Ltd., Japan). WO 2005085267.
Hyland K A, Kohrt L, Vulchanova L, Murtaugh M P. Mucosal innate immune response to intragastric infection by Salmonella enterica serovar Choleraesuis. Mol. Immunol. 2006 April; 43(11):1890-9.
Khan M A, Ma C, Knodler L A, Valdez Y, Rosenberger C M, Deng W, Finlay B B, Vallance B A. Toll-like receptor 4 contributes to colitis development but not to host defense during Citrobacter rodentium infection in mice. Infect Immun. 2006 May; 74(5):2522-36.
La Ferla B Cardona F, Perdigao I, Nicotra F. Direct Synthesis of Glycidic Bicyclic Scaffolds in Water without Protecting Groups SynLett 2005, 2641-2642.
Lo B., Silverman M., 1998, J. Biol. Chem. 273, 29341-29351.
Laukoetter M G, Nava P, Nusrat P. Role of the intestinal barrier in inflammatory bowel disease. World journal of Gastroenterology. 2008; 21,14: 401-407.
Mankertz J, Schulzke J. Alterated permeability in inflammatory bowel disease: pathophysiology and clinical implications. Curr Opin Gastroenterol 2007; 23: 379-383.
Motobu M, Amer S, Koyama Y, Hikosaka K, Sameshima T, Yamada M, Nakamura K, Koge K, Kang C B, Hayasidani H, Hirota Y. Protective effects of sugar cane extract on endotoxic shock in mice. Phytother Res. 2006 May; 20(5):359-63.
Nomura, S.; Kawanishi, E.; Ueta, K. Preparation of glycosides as antidiabetic agents and having inhibitory activity against sodium-dependant transporter. (Tanabe Seiyaku). WO 2005012326.
Nomura, S.; Kawanishi, E.; Ueta, K. Preparation of glycosides as antidiabetic agents and having inhibitory activity against sodium-dependent transporter. (Tanabe Seiyaku). US 2005233988.
Raja M. M., Tyagi N. K., Kinne R. K. H., 2003, J. Biol. Chem. 278, 49154-49163
Raja M. M., Kipp H., Kinne R. K. H. 2004, Biochemistry 43, 10944-10951.
Strassheim D, Park J S, Abraham E. Sepsis: current concepts in intracellular signaling. Int J Biochem Cell Biol. 2002 December; 34(12):1527-33.
Strober W, Fuss I, Mannon P. The fundamental basis of inflammatory bowel disease. The journal of clinical investigation. 2007, vol. 117; 3.
Troeger H, Epple H J, Schneider T, Wahnschaffe U, Ullrich R, Burchard G D, Jelinek T, Zeitz M, Fromm M, Schulzke J D. Effect of chronic Giardia lamblia infection on epithelial transport and barrier function in human duodenum. Gut. 2006 August 2510.1136.
Vincent J L. Management of sepsis in the critically ill patient: key aspects. Expert Opin Pharmacother. 2006 October; 7(15):2037-45.
Wills-Karp M. Interleukin-13 in asthma pathogenesis. Immunol Rev. 2004 December; 202:175-90.
Xia X., Lin C. T., Wang G., Fang W., 2004, Arch. Biochem. Biophys. 425, 58-64.
Yonekubo, S.; Fushimi, N. Preparation of 1-substituted-3-β-D-glucopyranosylated nitrogenous heterocyclic compounds as inhibitors of SGLT1 (sodium-dependent glucose transporter 1) and SGLT2. (Kissei Pharmaceutical). WO 20060526.
Zimmermann N, Hershey G K, Foster P S, Rothenberg M E. Chemokines in asthma: cooperative interaction between chemokines and IL-13. J Allergy Clin Immunol. 2003 February; 111(2):227-42; quiz 243).

1. Compounds of the general formula (I) and (II) 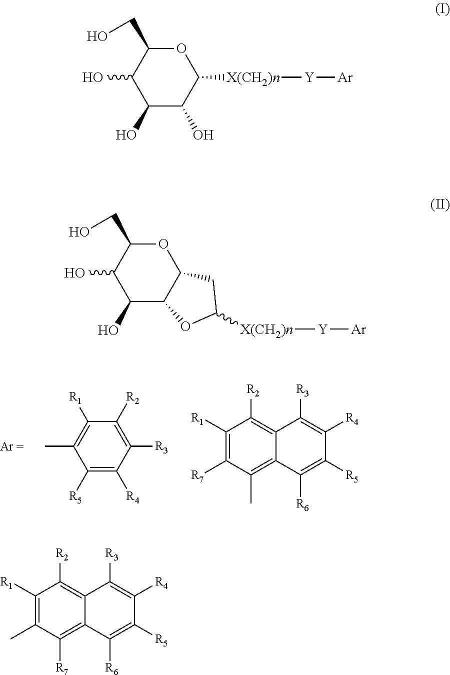
wherein
X=—CH₂—, —O—, —S—
n=0-10
Y=—NH—, —NHSO₂—, —NHSO—, —NHCO—, —S—, —O—, —CH═CH—
R₁-R₇, equal or different can be
hydrogen, alkyl C₁-C₄, alkenyl C₂-C₄, cycloalkyl C₃-C₇, aryl or heteroaryl;
may be substituted with one or more alkyl C₁-C₄, alkoxyl C₁-C₄, alkylthio C₁-C₄ or halogens;
—NR₈R₉, where R₈ and R₉, equal or different, represent hydrogen, alkyl C₁-C₄, alkenyl C₂-C₄, cycloalkyl C₃-C₇, aryl or heteroaryl, may be substituted with one or more C₁-C₄, alkoxyl C₁-C₄, alkylthio C₁-C₄ or halogens;
—(CH₂)_{n′}—COOR₁₀, with n′=0-4 and R₃=hydrogen or alkyl C₁-C₄,
and their enantiomers, their diastereoisomers, their addition salts with organic and inorganic acids, or their alkaline and alkaline earth metal or their ammonium ions,
with the exclusion of the compounds having the following formulae:

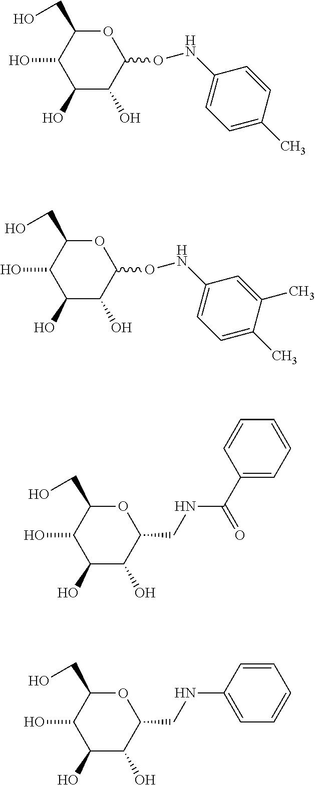

2. Compounds of formula (I) or (II) according to claim 1, wherein X represents CH₂.

3. Compounds of formula (I) or (II) according to claim 1 wherein n represents 0-5.

4. Compounds of formula (I) or (II) according to claim 1 wherein Y represents the group —NHSO₂— or —NH—CO—.

5. Compounds of formula (I) or (II) according to claim 1 wherein each of R₁-R₇ represent, independently of one another, a hydrogen atom or a —NR₈R₉ group.

6. Compounds of formula (I) or (II) according to claim 5 wherein R₈ and R₉ represent alkyl C₁-C₄.

7. Compounds of formula (I) or (II) according to claim 1, which are:
5-(dimethylamino)-N-[2′-(α-D-glucopyranosyl)ethyl]-1-naphthalenesulfonamide;
N-[2′-(α-D-glucopyranosyl)ethyl]-1-naphthalenesulfonamide;
N-[2′-(α-D-glucopyranosyl)ethyl]-1-naphthalenecarboxyamide;
5-(dibutylamino)-N-[2′-(α-D-glucopyranosyl)ethyl]-1-naphthalene sulfonamide;
5-(dimethylamino)-N-[2′-(α-D-galactopyranosyl)ethyl]-1-naphthalene sulfonamide;
5-(dimethylamino)-N-[4′-(α-D-glucopyranosyl)butyl]-1-naphthalene sulfonamide.
and their addition salts with organic and inorganic acids, or their alkaline and alkaline earth metal or their ammonium ions.

8. Pharmaceutical compositions comprising as active ingredient a compound according to claim 1 in combination with one or more inert, non-toxic, pharmaceutically acceptable carriers.

9. Pharmaceutical compositions according to claim 8, for the therapy of diarrhoea due to bacterial infections at luminal level, endotoxic shock, asthma, Crohn's disease, mucositis due to pharmacologic and radiotherapeutic treatments, diabetes mellitus.

10. Pharmaceutical compositions according to claim 8, possibly associated with other active principles.

11. Pharmaceutical compositions according to claim 10 for the therapy of endotoxic shock containing as active principle an association of 5-(dimethylamino)-N-[2′-(α-D-glucopyranosyl)ethyl]-1-naphthalene sulfonamide and glutamine.

12. Pharmaceutical compositions with hypoglycemic activity, according to claim 8, possibly associated with other active principles

13. Pharmaceutical compositions according to claim 12, for the therapy of diabetes mellitus.
